(19) United States
(12) Patent Application Publication
(10) Pub. No.: US 2026/0094696 A1
(43) Pub. Date: Apr. 2, 2026

(54) Reconstruction of Magnetic Resonance Imaging (MRI) Images from Accelerated Undersampled MRI Scans Using Machine Learning

(71) Applicant: Foqus Technologies Inc., Kitchener (CA)

(72) Inventor: Sadegh RAEISI, Kitchener (CA)

(73) Assignee: Foqus Technologies Inc., Kitchener (CA)

(21) Appl. No.: 19/409,384

(22) Filed: Dec. 4, 2025

Related U.S. Application Data
(63) Continuation of application No. PCT/CA2024/051484, filed on Nov. 8, 2024.
(60) Provisional application No. 63/599,839, filed on Nov. 16, 2023.

(51) Int. Cl. G16H 30/40; G06N 3/0475; G06N 20/00; G06T 5/50; G06T 12/20

(52) U.S. Cl. CPC G16H 30/40; G06N 3/0475; G06N 20/00; G06T 5/50; G06T 12/20; G06T 2207/10088; G06T 2207/20221

(57) ABSTRACT

A method for training machine learning models for making medical images such as magnetic resonance imaging (MRI) images from accelerated MRI scans that are shorter compared to standard scans. The method includes techniques for evaluations of the performance of the model which also provides new metrics and loss functions for training the models. The method also involves a technique for new designs for the machine learning models that combines the advantages and the best of different machine learning models for this application. The method also contains a step for fine-tuning task and condition-specific models that outperforms the performance for any generic model that is trained for all conditions and applications. The method also has a method for expanding the dataset used for training the models by generating new data samples and augmenting the data.

### CROSS-REFERENCE TO RELATED APPLICATION(S)

This application claims priority to U.S. Provisional Patent Application No. 63/599,839 filed on Nov. 16, 2023, the contents of which are incorporated herein by reference.

### TECHNICAL FIELD

The following generally relates to reconstructing MRI images from accelerated and undersampled MRI scans and, more particularly, to methods of training machine learning models for such reconstructing, and to evaluating the performance of the models and the reconstructed images.

### BACKGROUND

MRI is often considered to be the “gold standard” of medical imaging. MRI uses magnetic fields and radio waves to create detailed images of the organs and tissues in a subject's body. MRI can help diagnose a variety of health conditions, such as brain tumors, heart problems, spinal cord injuries, and more. MRI is not invasive, unlike X-ray or CT scans, so it is considered a safe choice for imaging.

A known bottleneck with MRI is that the scanning process is slow and typically takes too long to complete, which has been found to limit the access to MRI. One way to accelerate the process is to undersample the scans as shown in FIG. 1

However, the quality of the images declines with more undersampling and there is a limit to how much undersampling can be done before the MRI images lose their diagnostic capabilities.

Other, different techniques, such as compressed sensing (CS) and parallel imaging (PI) have been developed to improve the quality of the images reconstructed from undersampled scans.

CS techniques have been widely used in MRI reconstruction to accelerate the acquisition time and improve image quality. CS is a mathematical framework that reconstructs data from highly undersampled measurements. By exploiting the sparsity of MRI images, CS can reconstruct high-quality images from a reduced number of measurements.

PI uses multiple receiver coils to collect data and uses methods to unwind the signal and generate high-quality images. For both PI and CS, the quality of the images decline as more undersampling is done.

In recent years, machine learning (ML) techniques have been used as an alternative or a complementary technique to reconstruct high-quality images from undersampled scans.

There are numerous different ML techniques developed for this purpose, each of which has its own advantages and shortcomings.

### SUMMARY

The following describes a method for combining different models and fine tuning the models for specific applications and conditions. Further, methods for benchmarking and characterization of the performance of the models and the reconstructed images from them are also provided.

In one aspect, there is provided a method of training machine learning models for processing medical images, the method comprising: obtaining at least one loss function built using at least one evaluation technique to train machine learning models to process medical images; combining a plurality of image processing techniques using one or more ensemble techniques; building at least one task-specific model based on the one or more ensemble techniques and using the at least one loss function, to optimally perform on a corresponding specific type of image processing task or for a corresponding specific application or condition; and providing the at least one task-specific model to a system that performs the specific type of image processing task or the corresponding specific application or condition.

In certain example embodiments, the at least one evaluation technique comprises one or more of: i) at least one comparison metric; ii) diagnostic performance metrics to measure performance of the machine learning models or image processing task for the corresponding specific diagnostic application; iii) difference metrics to characterize the difference between the ground truth and processed images by building a difference map and measuring the information contained in the difference map; and iv) structural fidelity to measure how well structures are preserved in an image processed by the machine learning models.

In certain example embodiments, the at least one evaluation technique further comprises at least one quality metric.

In certain example embodiments, the at least one comparison metric comprises structure SSIM, informationally weighted SSIM, or Grad-SSIM.

In certain example embodiments, the at least one evaluation technique is applied to a foreground of images being processed by ignoring a background of the images being processed.

In certain example embodiments, the one or more ensemble techniques comprises stacking, boosting, or bagging.

In certain example embodiments, the image processing is optimized over a foreground of images being processed by skipping a background of the images being processed.

In certain example embodiments, the image processing comprises reconstructing medical images from accelerated image scans.

In certain example embodiments, the reconstructing utilizes machine learning (ML).

In certain example embodiments, the image scans correspond to a magnetic resonance imaging (MRI) scan.

In certain example embodiments, the specific type of image processing task or the specific application or condition comprises task-specific or tailored models for any one or more of: i) an MRI protocol, ii) a specific vendor or MRI machine, iii) an MRI scan view type, iv) a field strength, or v) an identified application or condition category, or vi) different acceleration rate.

In certain example embodiments, the method further includes utilizing synthetic data to augment the training.

In certain example embodiments, the synthetic data simulates the physical processes of (a) the pulses transmitted by the MRI machine and (b) the response electromagnetic pulses generated by the molecules and tissues in the body and collected by the MRI machine.

In certain example embodiments, utilizing the synthetic data comprises one or more of the following ways: i) synthesizing samples using MRI images and coil sensitivity maps from real data with a k-space, ii) using synthetic data that is generated using simulators and phantoms generated from MRI images, iii) using synthetic data that is generated using generative models to generate synthetic samples with raw data from the k-space, iv) using generative models to synthesize samples with a specific pathology or attributes; or v) augmenting existing samples both at a k-space level and an image level.

In certain example embodiments, the generative models comprise a Generative Adversarial Network (GAN) or style-GAN model.

In certain example embodiments, the image processing task comprises MRI motion correction or MRI image enhancement.

In certain example embodiments, the method further includes applying the method to a task related to x-ray, CT scan, or PET imaging.

In certain example embodiments, the method further includes applying the method to synthesize new samples for at least one imaging application.

In certain example embodiments, the at least one imaging application comprises MRI, x-ray, CT scan or PET imaging.

In certain example embodiments, the method further includes executing the method to process at least one medical image.

In another aspect, there is provided a computer readable medium comprising computer executable instructions that, when executed, cause a computing device to perform the method according to any one of the aspects or embodiments described above.

In another aspect, there is provided a computing device comprising a processor and memory, the memory comprising computer executable instructions that, when executed by the processor, cause the device to perform the method according to any one of the aspects or embodiments described above.

### BRIEF DESCRIPTION OF THE DRAWINGS

Embodiments will now be described with reference to the appended drawings wherein:

FIG. 1 illustrates undersampling using an accelerated MRI scan.

FIG. 2 illustrates labeling applied to an example scan.

FIG. 3 illustrates a machine learning pipeline.

FIG. 4 illustrates the application of diagnostic metrics.

FIG. 5 illustrates a difference map.

FIG. 6 provides an example of a workflow illustrating a structural fidelity benchmarking technique.

FIG. 7 illustrates an example of an architecture of an ensemble model with stacking.

FIG. 8 provides an example of a boosting architecture.

FIG. 9 provides an example of an architecture combining boosting and stacking.

FIG. 10 provides a schematic architecture of an overall model, containing task-specific models.

FIG. 11 is a flowchart illustrating an algorithm for selecting from a model archive in an example with two models.

### DETAILED DESCRIPTION

The following describes methods for training and evaluation of machine learning models for the processing or enhancement of medical images, for example, MRI, PET, CT scan, x-ray. While certain examples below are provided in the context of MRI imaging and tasks such as image reconstruction, the principles discussed herein can be equally applied to other types of images and image processing tasks, for example image restoration or image compression.

For the sake of illustration, the following describes a method for reconstruction of MRI images from accelerated MRI scans. However, the method described herein can be applied to other medical image modalities and other image processing applications.

The methods can be applied to both accelerated undersampled raw MRI data (k-space) or final images from accelerated undersampled MRI scans. In other words, the methods described here can be used to train and evaluate models that either start from accelerated undersampled raw MRI data (k-space) or start from final MRI images that are processed accelerated undersampled MRI scans.

The first step of the method involves training an ensemble of ML models for the reconstruction of the MRI images from undersampled raw data. These for instance could include architectures such as Varnet [1], LPDNet [2] and RecurentVarnet [3].

These models may all be trained as supervised models, on a dataset where the input (X) contains the raw data from accelerated undersampled MRI scans (k-space) and the target output (Y) are the MRI images from standard non-accelerated and fully sampled scans. Examples of such datasets are FastMRI [4] and Calgary Dataset [5].

The second step of the method involves applying ensemble ML techniques to the models in the ensemble.

One such approach is to train an aggregator-model such that it would combine the results of all the models in the ensemble to generate an MRI image that would have a better quality compared to the individual predictions of the models in the ensemble.

The models in the ensemble could be different architectures or the same architecture trained with different loss functions or the same architecture, trained under different conditions. For this step, different ensemble techniques may be used, including the stacking that was described above. Similarly, other ensemble techniques in ML, such as bootstrapping and boosting can be used.

In the case of bootstrapping, one can use different random subsets of the data to train the same architecture and then an aggregator model to combine the results of the models that are trained on different subsets of the data.

In the case of boosting, models can be sequentially trained for each architecture to enhance the quality of the model on subsets of the data that the model is under-performing on.

The third step of the method involves fine-tuning the trained model (or/and their ingredient models) for specific MRI scans (Axial, Sag, . . . ), protocols (T1, T2, Flair, . . . ), and conditions (vendors, machine type, field strength).

Finally, the trained models may be fine-tuned one last time for each specific MRI machine that the software is going to be deployed on.

The method can similarly be used for when instead of the k-space, the inputs are images generated from the MRI scans instead of the raw data.

A computer readable medium can be produced that includes computer executable instructions that, when executed, cause a computing device to perform the method to train the models.

A computing device including a processor and memory can also be produced or programmed such that the memory includes computer executable instructions that, when executed by the processor, cause the device to perform the method to train the models.

The following also includes techniques for quantifying the quality of ML models and the reconstructed images, which have been recognized to be particularly suitable when applied to medical imaging for MRI, as discussed herein. These May include new metrics such as informationally-weighted SSIM [6] and Gradient SSIM [7, 20]. The method can also include evaluation techniques that assess the down-stream and diagnostic capabilities of the images generated by the models. The method can also incorporate techniques for augmenting and synthesizing data for training models.

For medical imaging with MRI, it is recognized that there is usually a trade-off between the quality of the MRI images and scan time. Ideally, one desires to have high-quality and detailed images acquired with fast scans. But, usually, for fast scans, the quality of the images is compromised and degraded. Alternatively, to get high-quality images, scan time should increase.

Techniques such as PI and CS provide solutions that overcome this to some extent [8, 9] as noted above. ML techniques have attracted a lot of attention recently as a technique for reconstruction of high-quality images from extremely undersampled and fast scans.

The following proposes a method that can significantly improve upon these current techniques.

This presents a method for training machine learning models for reconstruction of MRI images from undersampled MRI scans, either in the raw format (k-space) or image data and for evaluation of the models. The current method provides techniques for combining the benefits of different models of MRI image reconstruction and provides superior image quality from faster (more under-sampled) scans.

For any MRI scan, there are two elements, the raw data (often referred to as the “k-space”) and the MRI image. The raw data is the data that is collected by the MRI machine. The MRI image is the result of some processing applied to the raw MRI data. Herein, MRI images may be referred to as “MRI-IMG” and the MRI raw data referred to as “MRI-KS”.

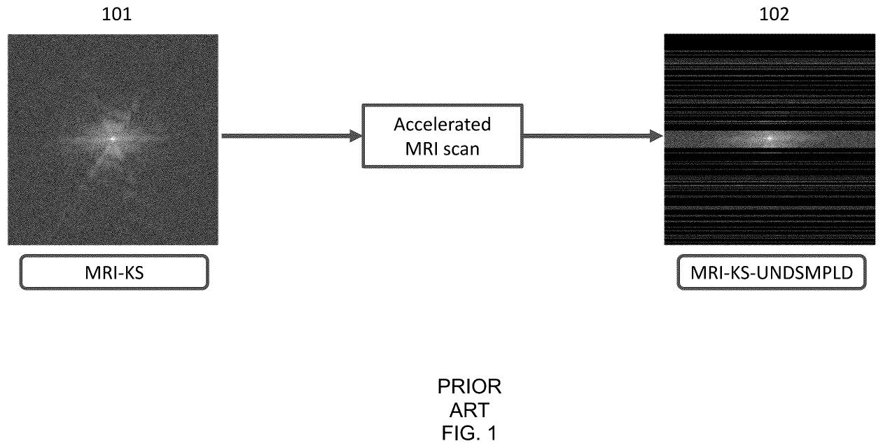

Furthermore, for this disclosure, the proposed methods can deal with two types of MRI scans, namely fully sampled MRI scans (i.e., the standard scans) and accelerated MRI scans (i.e., undersampled scans). One may refer to the standard scan with a tag of “STNRD” and to the accelerated scans as “UNDSMPLD”. For instance, for the raw data from an accelerated scan, one can use the label MRI-KS-UNDSMPLD (e.g., as labeled in the rightmost image of FIG. 1). Each MRI scan involves collecting a sequence of rows (which corresponds to different phases of the electromagnetic waves) and concatenating them in a matrix like the left image 101 in FIG. 1. This is referred to as the raw data or the k-space. To accelerate the MRI scan, the scan is undersampled, meaning that only a subset of all the rows is collected. The right image 102 in FIG. 1 shows the result of such setting. The black rows indicate the row or phases that were not collected. The scan corresponding to the right figure is faster because there are fewer rows (phases) collected. The more the k-space is undersampled, the more the scan is accelerated.

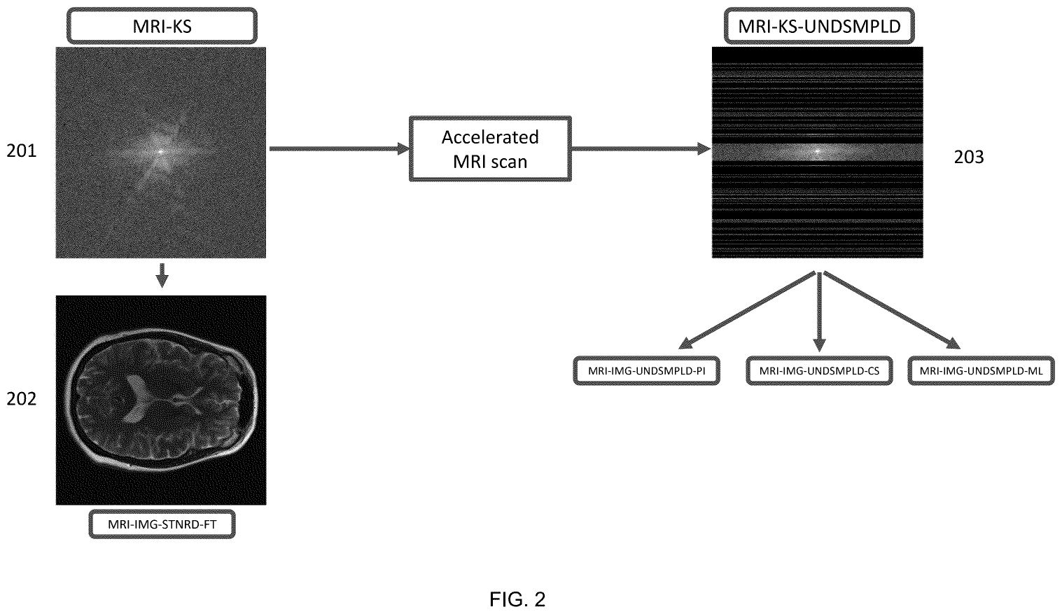

For the MRI images (i.e., MRI-IMG), one may also use a tag to describe how the images are reconstructed and processed from the raw data (i.e., MRI-KS). For this one can use “FT” for the standard reconstruction technique that uses a Fourier Transformation, “CS” for compressed sensing-based reconstruction, “PI” for parallel imaging and “ML” for machine learning based reconstruction. As such, for an image reconstructed from an accelerated scan using ML, one can use the label “MRI-IMG-UNDSMPLD-ML”. FIG. 2 shows these labels for a sample scan. This figure shows the different elements of the data and image processing for an MRI scan with notations/labels/terminology used in this disclosure. On the top left image 201, is the standard fully sampled k-space (MRI-KS). On the bottom left image 202 is the MRI image generated from the standard fully sampled k-space (MRI-KS). This is generated by applying an inverse Fourier transformation (and in the case of multi-coil scans, averaging) on the MRI-KS and is referred to as MRI-IMG-STNRD-FT. The undersampled (accelerated) k-space 203 is referred to as MRI-KS-UNDSMPLD. It is possible to apply the inverse Fourier transform (IFT) to MRI-KS-UNDSMPLD to get an image, which may be referred to as MRI-IMG-UNDSMPLD-FT and which is often blurry and low quality. There are other techniques that can generate better images. The include using parallel imaging (PI), compressed sensing (CS) and ML. One can tag the MRI image generated from the undersampled scan with the technique that is used to generate the image. That is MRI-IMG-UNDSMPLD-PI for images reconstructed with PI, MRI-IMG-UNDSMPLD-CS, for images reconstructed with CS and MRI-IMG-UNDSMPLD-ML for images reconstructed ML.

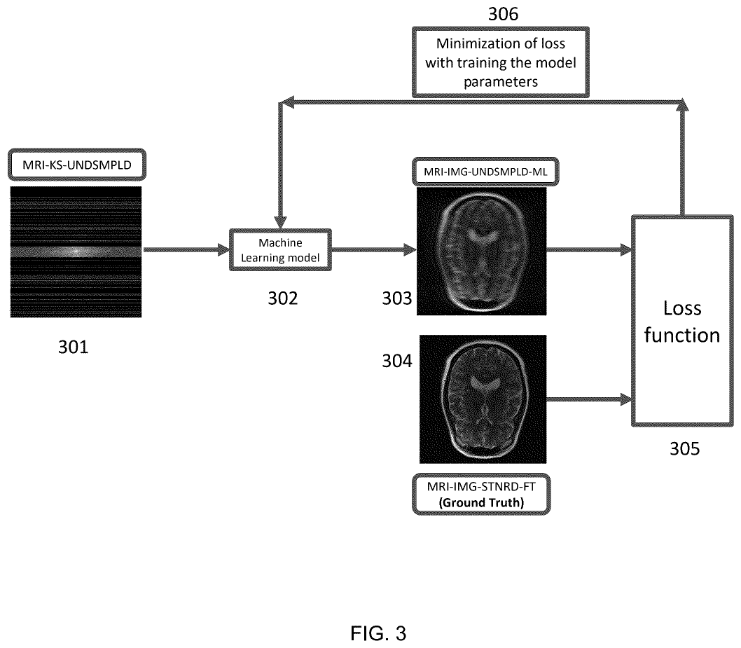

FIG. 3 provides a schematic and high-level description of the ML pipeline. The task of the model is to reconstruct MRI images from accelerated (undersampled) MRI scans with a quality that would match the standard MRI scans, that is the level of quality that a standard MRI scan would have. This pipeline is a supervised learning pipeline which needs both an input and a target output (ground truth) for training. In this pipeline, the raw data collected from accelerated scans (i.e., MRI-KS-UNDSMPLD) are fed to the machine learning model as an input and the ML model generates MRI images as the output. Specifically, this figure shows the pipeline for a machine learning model for MRI image reconstruction from an input undersampled MRI scans 301. The model 302 generates an image. This image is compared against the ground truth 304 which is the image from the standard fully sampled MRI scan. This comparison is quantified using a loss function 305. The loss function May be a mathematical function on the predicted image 303 and the ground truth 304. It may also involve a procedure that associates a number to the distance between the two images. The goal is to adjust the parameters of the model such that the loss function (distance between the predicted image and the ground truth) is minimized. This is known as training the model. Once the loss function is calculated, an optimizer 306 is used to minimize the loss function by training the model.

For training the ML models in this pipeline, the system uses a dataset with both the input and the target output. The inputs are MRI-KS-UNDSMPLD. The target outputs are standard MRI images, i.e., MRI-IMG-STNRD-FT. 6

There are typically two ways to collect such a dataset. One can run both a standard MRI scan as well as an accelerated MRI scan on the subjects. However, this is normally expensive and takes longer for the patients. Also, it needs to be repeated for any specific amount of acceleration if the objective is to build and evaluate models for different levels of acceleration.

Alternatively, it is possible to only collect the standard fully sampled scan and simulate accelerated scans by undersampling the MRI-KS-STNRD. The system can use this technique to provide more flexibility for making different types of undersampling.

The goal is for the output MRI images generated by the ML model (MRI-IMG-UNDSMPLD-ML) to be identical or as close as possible to the standard MRI images (MRI-IMG-STNRD-FT).

For this, the system first defines techniques, measures and metrics for evaluation of the quality of the output images. Then one can define loss functions for training the ML model such that optimizing the loss function (i.e., training the model) would ensure that the MRI-IMG-UNDSMPLD-ML has high quality and is a good fit to MRI-IMG-STNRD-FT according to the defined comparison techniques, metrics and measures.

### Evaluation Metrics, Measures and Techniques

One can consider different approaches for evaluating the outcomes of ML models. These can be categorized into different categories.

First, there are the measures that characterize the quality of the output image (MRI-IMG-UNDSMPLD-ML) without any reference and purely on its own. The main metric for this is the Signal-to-Noise-Ratio (SNR), referred to as “quality metrics”.

Second, there are the metrics for comparing the MRI-IMG-UNDSMPLD-ML and MRI-IMG-STNRD-FT. One example in this category is the Structural Similarity Index Metric (SSIM), referred to as “comparison metrics”.

Third one can use techniques that measure the quality of the output images based on their performance towards specific diagnostic applications, referred to as “diagnostic performance metrics”.

Fourth, one can use techniques that characterize the difference between the reconstructed MRI images and the ground truth and the information missing in the reconstructed images, referred to as “difference metrics”.

The fifth category of benchmarking techniques involves adding artifacts to the MRI images and measuring how well they are preserved during the reconstruction process, referred to as “structural fidelity”.

The metrics are mathematical functions that are used to quantify how well a reconstruction is or how well an ML model performs. With a metric, the higher the value, the better the performance is.

The metrics can be used to build loss functions that characterize the dissimilarities between the predicted MRI images and GT images. The loss functions are important because they are needed for training the ML models. For instance, the SSIM ranges between −1 and 1 with 1 indicating perfect similarity, 0 indicating no similarity and −1 indicating anti-correlation. To turn this into a loss function, one can use:

$${L}_{SSIM} = 1 - \vert SSIM \vert$$

For all of the techniques introduced here, it is possible to go back and forth between the metric and their corresponding loss function.

One may also refer to some of the techniques introduced here as benchmarking techniques or the evaluation techniques which would include the method used for calculating the loss and metric function.

Metrics introduced here can be used beyond the applications of MRI image reconstruction with ML models and can be used to quantify the performance for any MRI image or any (medical) image processing tools. For instance, they can be used for CT, PET scan images or to evaluate motion correction on medical images.

In this technique, contrary to common methods, the present system does not use only the average of the score over a test or validation set for performance characterization. The system also uses standard deviation (SD) as well as worst-case, 95% and 99% value.

The 95% indicates the score that the performance of the 95% or more of the samples or MRI images fall above that score. This value can be set based on the specifics of the problem.

One can also use score histograms to characterize how the samples and their corresponding scores are distributed.

i. Quality Metrics

As indicated above, quality metrics may refer to existing measures that characterize the quality of the output image (MRI-IMG-UNDSMPLD-ML) without any reference and purely on its own.

ii. Comparison Metrics

One of the most well-established metrics in this category is SSIM.

The system described herein enables different methods for the evaluation of the performance of the MRI images using different variations of SSIM. The current disclosure also describes/introduces a method for optimizing the ML models for getting better performance with respect to each of these metrics. It is recognized that using structure-SSIM, IWSSIM, and grad-SSIM for MRI reconstruction is a new approach that provides particularly good results, as discussed below. Moreover, it may be noted that in the application of these techniques, the system does not only look at the average of the score, but rather considers the worst case, and scores that a specified threshold of the samples that fall above.

### Structure-SSIM

The SSIM is a widely used metric for assessing the quality of images. It includes several elements that capture different aspects of image similarity and degradation. One of the elements of SSIM is the preservation of edge information between the reference and test images [16]. This element measures how well the edges in the test image match those in the reference image. Changes in edge information can indicate degradation in image quality. Another element of SSIM is the preservation of texture [16]. Texture refers to the patterns and details in an image. SSIM measures how well the texture in the test image matches that in the reference image. Changes in texture can also indicate degradation in image quality. The structural component of the images is another element of SSIM [16]. This element captures the overall structure and arrangement of objects in the image.

For the MRI images, the system is particularly concerned with the structure and arrangement of objects in the image. In other words, when one compares the output MRI images with the ground truth, it is ok if they do not perfectly match the brightness or texture of the ground truth. However, it is important for the structure of the output image to match the structure of the ground truth.

By structure-SSIM, the following refers to a SSIM where only the structural term is considered or its weight in calculation of the metric is emphasized.

The current method involves measuring the performance of the MRI images with respect to the ground truth using structure-SSIM. Also, for training ML models that optimize for structure-SSIM, the structure-SSIM is built into a loss function (similar to the one for SSIM) and then used to train ML models.

### Informationally Weighted SSIM (IW-SSIM)

Informationally weighted SSIM (IW-SSIM) is a full-reference image quality assessment method that incorporates information content weighting into the structural similarity index (SSIM) [17]. The goal of IW-SSIM is to provide a more accurate and perceptually meaningful measure of image quality by considering the importance of different image regions based on their information content.

Traditional image quality assessment methods, such as peak signal-to-noise ratio (PSNR) and SSIM, focus on measuring fidelity and similarity between the reference and distorted images [18]. However, these methods do not take into account the fact that different regions of an image may have varying levels of importance in terms of visual perception. IW-SSIM addresses this limitation by introducing a weighting scheme that assigns higher weights to regions with higher information content and lower weights to regions with lower information content [20].

The current method involves measuring the performance of the MRI images with respect to the ground truth using IW-SSIM. Also, for training ML models that optimize for IW-SSIM, the IW-SSIM is built into a loss function (Similar to the one for SSIM) and then used to train ML models.

### Grad-SSIM

Grad-SSIM, or “Gradient-Weighted Structural Similarity Index”, is a metric used for image quality assessment that incorporates gradient information into the calculation of structural similarity (SSIM) [19]. The purpose of Grad-SSIM is to provide a more accurate and perceptually meaningful measure of image quality by considering the importance of image gradients. In the context of image restoration, such as diffraction-degraded remote sensing images, a common artifact known as ringing can occur [19].

The current method involves measuring the performance of the MRI images with respect to the ground truth using Grad-SSIM. Also, for training ML models that optimize for Grad-SSIM, the Grad-SSIM can be built into a loss function (similar to the one for SSIM) and then used to train ML models.

iii. Diagnostic Performance Metrics

An important expectation from the reconstructed images is to perform at the same level as standard MRI images for diagnostic purposes. To this end, diagnostic performance metrics are utilized that measure the performance of the ML models with respect to specific diagnostic applications.

These specific diagnostic applications could be detection of cancer, dementia, Alzheimer's disease or other reasons for which an MRI may be prescribed.

This evaluation technique involves first using the standard MRI images for the diagnostic application. For instance, if the task is detection of cancer, the standard set of images in the test dataset are used to identify which ones are positive and which ones are negative.

For this diagnosis, one may use experts (e.g., radiologists) to review the standard MRI images (MRI-IMG-STNRD-FT) and decide if the cases are positive. Alternatively, one may use secondary ML models that are designed and trained for the specific task (e.g., cancer detection) from MRI images [15].

The diagnosis of the samples in the test dataset from the standard images provides the ground truth for these samples. In other words, the system described herein uses the diagnosis from the standard image as reference/ground truth.

Next, the system runs the same test using the reconstructed MRI images (MRI-IMG-UNDSMPLD-ML). More specifically, one can get radiologists (or secondary ML models) review the reconstructed MRI images for the specific task (e.g., cancer detection) and predict their diagnosis from these images.

Now, the system can use the predictions from the images reconstructed by ML models (MRI-IMG-UNDSMPLD-ML) and compare them against the ground truth extracted from the standard MRI images (MRI-IMG-STNRD-FT).

This enables the system to use typical classification metrics such as accuracy, precision and recall to evaluate the performance of the reconstructed images for the ML model for that specific task (e.g. diagnosis of cancer).

With this method, it is also possible to calculate the confusion matrix for the predictions/diagnosis that was done based on using the reconstructed images with the machine learning models.

It may be noted that one can use the prediction/diagnosis from standard MRI images (MRI-IMG-STNRD-FT) as ground truth, but in some cases, these diagnoses might also be wrong. Specifically, there is some research on the capabilities of standard MRI images for revealing different pathologies and they are not perfect and are especially susceptible to false negatives.

Here the example of cancer detection has been considered. However, the method described here for measuring the performance of the model is not limited to cancer detection and can be extended to all other applications of MRI. For segmentation applications, the same method can be used in combination with metrics used for assessing the performance of segmentation (e.g. Dice coefficient).

It may also be noted that the applications of this method are not limited to evaluation of the performance of ML models that are built for reconstruction of MRI images from accelerated MRI scans. The same method can be applied to any other MRI image enhancement and MRI image modification technique/tool. For instance, the method described here can be used to assess the performance of solutions that are designed to correct motion artifacts in MRI images.

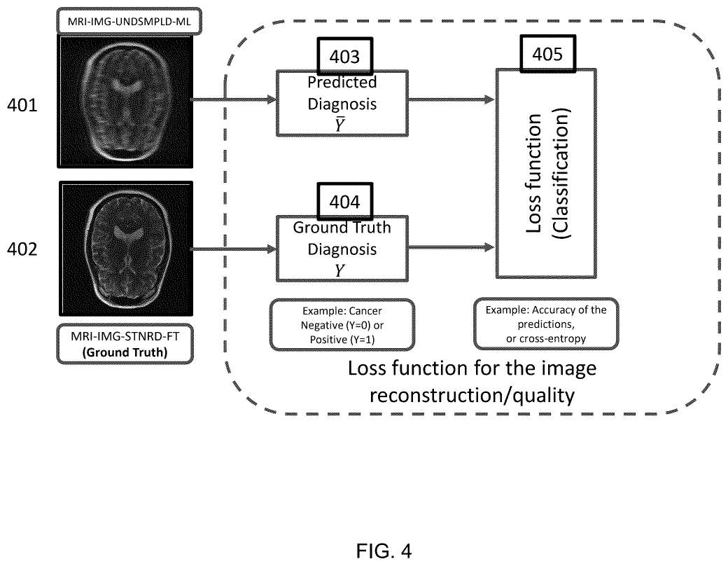

FIG. 4 illustrates a schematic of a diagnosis performance metric as herein described. This figure shows how the diagnostic metrics work. The MRI image predicted by the ML model needs to be compared against the ground truth 402. To this end, the two images are used towards a specified diagnosis task. This may be cancer detection, for instance. This generates a prediction from each image. The predication from the predicted image 401 is referred to as Y̅ 403 and the predictions from the ground truth 402 are labeled as Y 404. For instance, for a cancer detection diagnosis task, the Y̅ and Y can be negative (0) or positive (1). The Y̅ and Y can be extracted by a radiologist or even with a secondary machine learning model that is designed and trained for cancer detection. Once the predicted and the ground truth images 402 are mapped to Y̅ and Y, standard classification metrics (such as accuracy, recall, precision, . . . ) and classification loss functions 405 (such as cross-entropy) can be used to quantify the difference between the predicted and ground truth images 402.

One can also use these evaluation techniques to train models that optimize their diagnostic powers of the models. For this, one first trains a model for the specific diagnostic task (e.g., cancer detection). Then the system can concatenate the secondary model to the image reconstruction model and fix the weights of the secondary model. In this way, training models with classification tasks such as accuracy or cross-entropy would train the model to reconstruct images that preserve the diagnostic property of the MRI scans for that specific pathology. This can also be turned into a loss function.

Now that the system has identified the techniques and metrics for quantification of the models, one can use them to evaluate the results of the models that are trained and optimize the models to achieve high scores with these metrics.

It may be noted that these metrics can be used independent of the ML model and the training pipeline. In other words, the evaluation techniques in this disclosure can be used for the evaluation of the quality of MRI images. These techniques may also be used for other reconstruction techniques. They also may be used for evaluation of other tools and techniques that deal with quality of MRI images. For instance, there are tools for MRI motion correction, MRI image enhancement and post-processing. These techniques in this disclosure can be used for characterization of the quality of these techniques as well.

iv. Difference Metrics

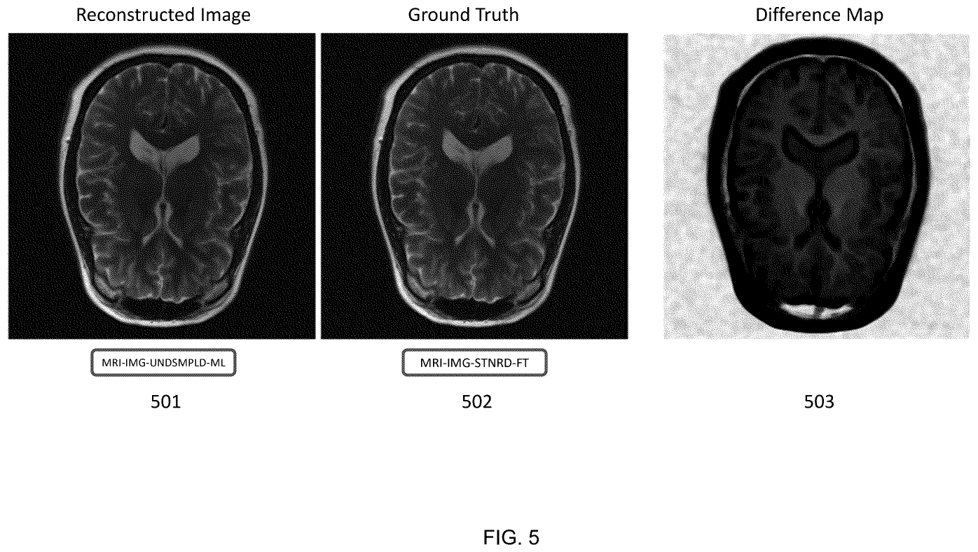

For the difference metrics, the system first subtracts the reconstructed image from the ground truth and a difference map and calculates the absolute values of the result of the subtraction, referred to herein as the “Difference Map (DM)”. FIG. 5 shows an example of a difference map. This figure shows the predicted or reconstructed MRI image 501, the ground truth 502 and the difference map 503. The difference map 503 characterizes the pixel-by-pixel difference between the predicted and ground truth images 502.

An important objective of difference metrics is to characterize how much information the difference map contains. Structures in the difference map can indicate missing structures in the reconstructed images. Ideally, the difference map should be zero, but this is found to rarely happen. As such, the system can try to make the reconstructions such that the values of the difference map are small and also not structured. There are different ways to characterize the information in the difference map. One example is to use the following formula as a loss function (with the smaller value the better).

$${L}_{DM} = \frac{{d}_{max}}{{ks}_{max}} H ( DM ) ,$$

where dₘₐₓ is the maximum value of the difference map (DM), the ksₘₐₓ is the maximum (absolute) value of the k-space tensors or data and H (DM) is the entropy of the difference map (DM) which is calculated using the Von Neumann Entropy.

The fraction in the equation is to normalize the difference map values to the values of the ground truth. If the difference values are too small compared to the values of the k-space data, this factor would suppress the loss value.

Similarly, if the difference map is random, H (DM) would be small and the metric would return a small value for the loss.

The system may use different variations of this loss function to characterize the difference map information. For instance, one may use a normalized difference map i.e.

$${DM}^{-} = \frac{DM}{{ks}_{max}}$$

instead. Similarly, one can use other functions instead of the Von Neumann Entropy.
v. Structural Fidelity

Ideally, the reconstruction should preserve the artifacts and structures in the MRI image. For instance, if there is an object (e.g., white disk) in the standard MRI image, it should be preserved in the reconstruction by the machine learning model or any processing that is done on the MRI image.

Normally it is not easy to do this because it is not easy to know what objects there are and it can be difficult to track how they change through the reconstruction or processing of the images.

The method in this disclosure includes a benchmarking technique that involves identifying or adding artifacts of different shapes and sizes to the MRI scans and then tracking the fidelity of these artifacts through the image reconstruction or image processing.

In the first approach, segmentation models can be used to specify different segments of the image. Then, the same process can be done with undersampled scans and two segmentations are compared to see how well they match. This indicates how well the structure of the image is preserved through the process.

The first approach can be quantified in different ways. For instance, one can calculate the Dice coefficient between the segmentation from the standard scan (MRI-IMG-STNRD-FT) and the segmentation from the reconstructed MRI image (MRI-IMG-UNDSMPLD-ML).

The second approach involves adding artifacts to the MRI scan and checking how these artifacts are preserved. This is directly related to diagnostic performance of the reconstructed MRI images.

For instance, to add the artifacts, one may start from the MRI fully sampled k-space, generate the MRI images, then add the artifact(s) and transform the image back to the k-space. Now, one can undersample the k-space data and apply the ML model to reconstruct the MRI images. Segmentation loss functions such as the Dice coefficient can be used to quantify how well the artifact is preserved.

The artifacts can be different shapes (disk, circle, oval, arc . . . ) and of different sizes (or classes of sizes, e.g. small medium or large). The artifacts can also be placed randomly at different parts of the image. Their intensity can also be different.

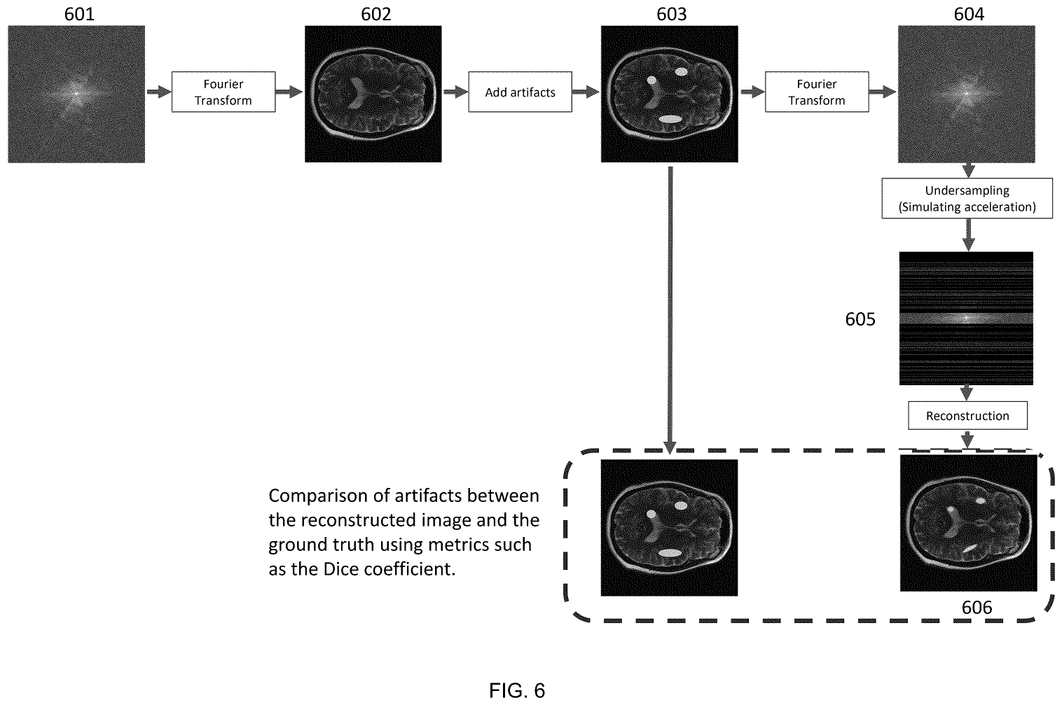

Moreover, it is possible to use generative models such as Generative Adversarial Network (GAN) models to add the artifacts to the models. In this approach, a generative (e.g., GAN) model would be trained to take an MRI scan and generate MRI scans with artifacts of different shapes, sizes and in different parts of the image. FIG. 6 shows the structural fidelity process schematically with example images. Specifically, this figure shows one example implementation of the structural fidelity technique for benchmarking reconstruction models. The input MRI scan data is transformed into an MRI image 602. Then, artifacts of random shape and sizes and positions are added to the MRI image 603. Next, the image with the artifacts is transformed back into the k-space 604. Then, this k-space data is undersampled 605 to simulate an accelerated scan. Finally, an image 606 is generated from the undersampled k-space data 605. The artifacts in the reconstructed image are compared against the image in 604 to check how well the reconstruction process has preserved these artifacts. For this comparison, different methods such as segmentation metrics (e.g. Dice coefficient) can be used.

### Model Architecture

Next, an example of implementing the ML pipeline is provided and how it may be trained.

### Ensemble Techniques for MRI Reconstruction

Different techniques, different ML models, and different architectures have been developed for the task of reconstruction of MRI images from accelerated MRI scans [1-3]. Each of these have their own strengths and weaknesses.

The current disclosure describes a method that combines different techniques and AI models to benefit from the strengths of the different techniques. The method in this disclosure leverages ensemble techniques, to combine different models.

For the sake of clarity, one can refer to the different ML models or other techniques that provide MRI images from accelerated MRI scans as “micro-models”. An example of a micro-model is Varnet [1].

An example of an ensemble technique used in this disclosure is based on Stacking [14], referred to as “stacking”. Also, the following may refer to the collection of the micro-models as the “ensemble”.

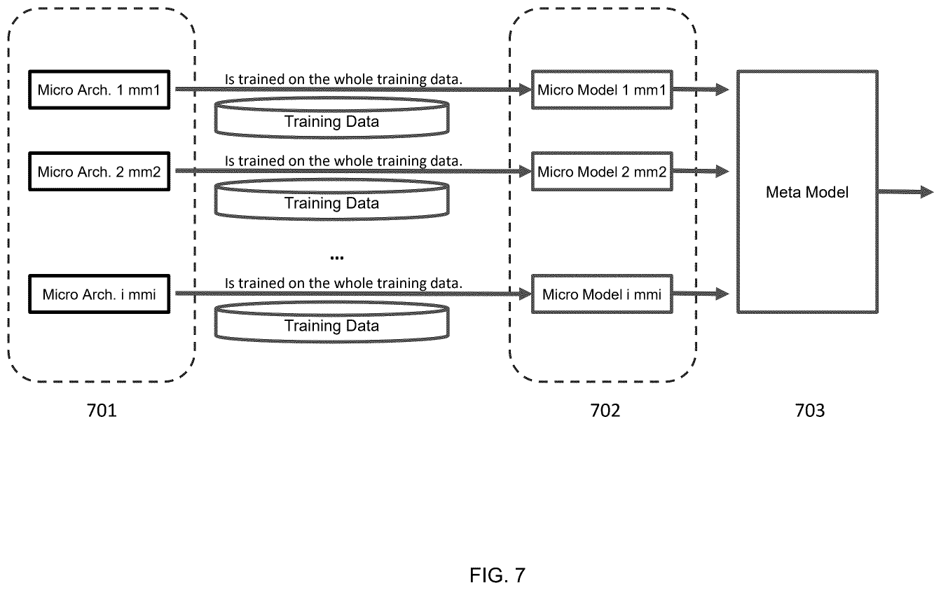

The stacking technique involves stacking the micro-models together. More specifically, this involves first training the micro-models. Then, the resultant micro-models are stacked together and a secondary model is trained to aggregate the results of the micro-model to generate a better MRI image (according to the metrics discussed above). The secondary model may be referred to as the “aggregator model”. Additionally or alternatively, this may, in some instances, be referred to as the “meta model”. FIG. 7 shows an example schematic of one such stacking architecture. This figure illustrates an example of how different models and algorithms for reconstruction of MRI images can be combined in a stacking ensemble architecture. It starts with an ensemble of model architectures 701. These are usually some of the state-of-the-art architectures that been reported in the literature and are expected to perform well. Next, the training data (or a subset of the training data) is used to train the models in the ensemble. This leads to an ensemble of trained models 702. Each of these models may already have a high performance. Then the system runs inference using the trained models in the ensemble to generate MRI images from the raw data from samples in the training data or for a subset of them. This maps each input k-space tensor to a sequence of MRI images, each generated by one of the models in the ensemble.

Next, the meta model 703 is trained. The input to the meta model is the sequence of the MRI images and the target is the original ground truth MRI image. Different architectures maybe used for the meta model (e.g. U-net). Also, for the meta model, it is possible to provide the completed (inferred k-space tensors) as additional input.

The inputs for the aggregator (or meta) model can be the MRI images that each micro-model generates. Alternatively, the reconstructed k-space from each 4 model can also be supplemented to the MRI images of each model. In other words, the input can be a combination of the k-space reconstruction of each micro-model as well as the MRI image generated from each micro-model.

The ground truth for the aggregator model is the image from the fully sampled MRI scan, i.e. MRI-IMG-STNRD-FT.

Different architectures can be used for the aggregator model, including but not limited to convolutional neural networks such as a UNET model and auto-encoder networks.

For the micro-models in the ensemble, it is possible to include the same architectures trained with different loss functions. For instance, the Varnet model trained with SSIM and the Varnet model trained with IW-SSIM can be included as two different micro models. This helps take advantage of the optimization with different loss functions.

Also, it is possible to include non-ML models in the ensemble. For instance, techniques such as compressed sensing or their variations, or parallel imaging and their variations (SENSE, SMASH) can be included in the ensemble as micro-models.

It may be noted that the aggregator model can be trained and tuned with a loss function different from the micro-models. For instance, the micro-models may be trained using SSIM and the aggregator can then be trained using mean-square-error (MSE) or a combination of SSIM and MSE (SSIM-MES).

The method can also involve using techniques similar to Boosting which is another ensemble ML technique. For instance, for a model, this involves training a primary model, e.g., micro-model-1. Then, the system can identify the set of samples that the model is not performing well on, referred to as “weak samples 1 (WS-1)”. Next, the system trains a secondary model (micro-model-2) that puts more emphasis on the samples in WS-1 to get better results. This can be done for instance by changing the loss function or including more copies of the WS-1 in the dataset used for the training. This process is repeated for several cascades, referred to as “Boosting”. The number of cascades can be determined either based on an expected performance score for the model or be predefined.

The result of the model can be a weighted average of the outcomes of the individual micro-models. Or, alternatively, a secondary (meta) model may be used to aggregate the outcomes of the models to get the output.

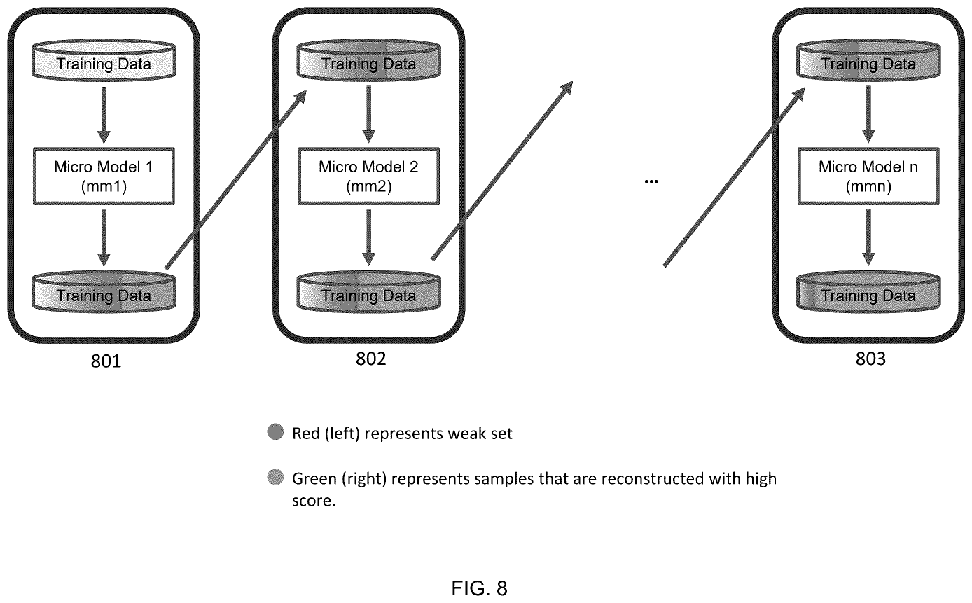

In contrast to stacking, this is a serial way of making an ensemble model with the micro-models to improve the performance of the overall model. FIG. 8 provides an example schematic of a boosting architecture. This figure illustrates an example of an ensemble technique that leverages boosting. In each step from left to right, a new micro model is trained which is focused on the samples in the dataset that the previous models in the sequence were not performing well on. For instance, in 801, the first model is trained. The red in the training data illustrates the subset for which the performance of the model is low. This is then fed to the second model at which puts more weight on the weak samples (red subset) and as a result, it reduces the weak set (smaller red subset). This process can continue until the model gets an acceptable performance over all samples (i.e., up to the Nth model at 803).

The system described herein can also involve using a technique similar to the Bagging machine learning techniques [11], referred to as “Bagging”. This is similar to stacking in structure, meaning that there is an aggregator model that aggregates the results of the micro-models in the ensemble. However, the micro-models are homogeneous, i.e., have the same architecture and trained on the same loss function. The micro-models for this approach are trained on different random subsets of the training dataset. This can help train micro-models that are each good for specific subcategories of the data. And then the aggregator combines the results of all the models in the ensemble, to make sure that the system takes advantage of the best micro-model for each subcategory.

For this approach, it is also possible to average the results of the micro-models or use a weighted average instead of training an aggregator-model.

Each of these ensemble techniques leverages a different aspect of the data and the model architectures that we have available. Stacking takes advantage of different architectures and techniques of reconstruction. Bagging benefits from micro-models tuned for different subcategories of whole data. Boosting, similar to Bagging, trains models that work well on subcategories of the data, but the design in sequential and the subcategories are chosen based on the set that the previous micro-models in the sequence were underperforming on (weak set) and tune the next-micro model in the sequence to do well on the weak set remaining from the previous micro-models.

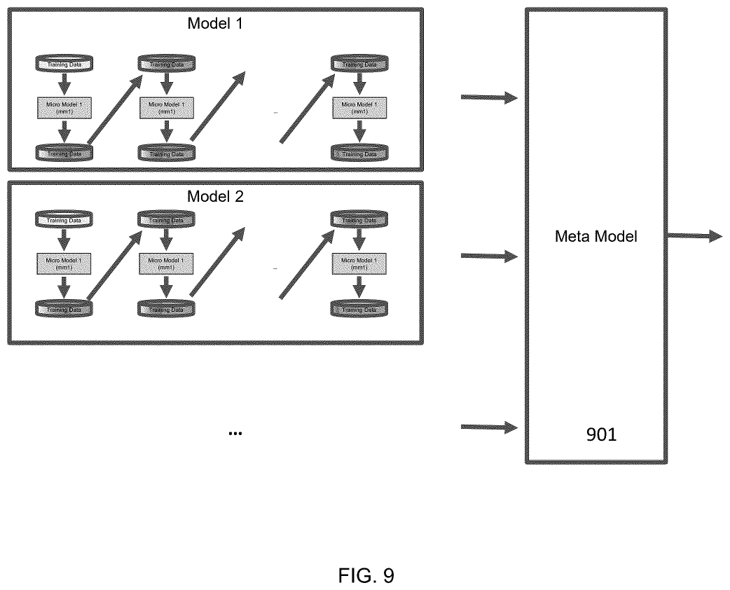

The method described herein can include combining these ensemble techniques (or their variations). For instance, one can use Bagging and/or boosting to train models based on each architecture that performs relatively well on different subcategories of the data. For this, one would repeat the Boosting process for different model architectures and then use the resultant models as a micro-model in an ensemble for stacking. FIG. 9 provides an example schematic of this technique. This figure illustrates an example architecture combining the boosting and stacking ensemble techniques. Each box on the left (labeled with model 1, model 2, . . . ) is itself a model that is built using boosting ensemble technique. Then the collection of these models (model 1, model 2, . . . ) are stacked together using a meta model 901 and build the whole model.

### Task-Specific Models

Another important element that can be utilized in the current method is fine-tuning the models for specific conditions and tasks. 2

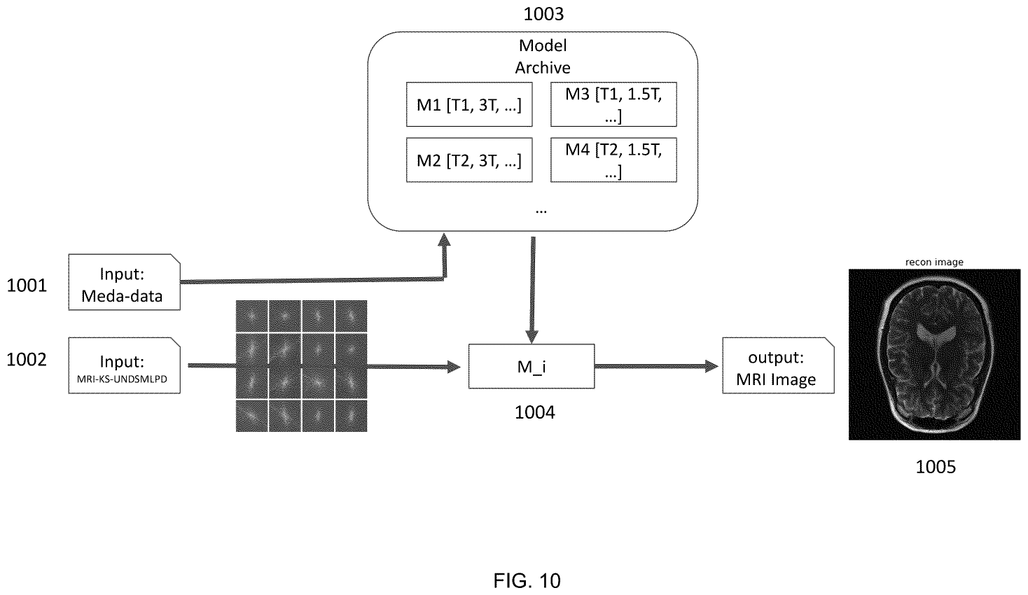

Instead of building one model that is used for all the different types of MRI scans, the method in this disclosure can involve training models specific to each application or condition. For instance, instead of using one model for T1, T2 and Flair images, a separate model is trained for each of them. More specifically, the method here can involve building a “model archive” where for each application/condition, there is a specific model. The method can include an algorithmic step where the case-specific model is picked based on the specifications of the input scan (tags from the DICOM file or the raw data). FIG. 10 shows a schematic architecture of the overall model, containing task-specific models. Each model in the ensemble is trained and constructed based on the design explained above. This figure shows how the inference pipeline works. The input has two elements, the meta-data 1001 and the k-space raw data 1002. The process of building the model involves building an archive of models 1003, each tuned for specific types or MRI scans or for specific conditions. For instance, the archive can include a model tuned specifically for Axial T1 scans from 1.5T GE scanners. The meta-data of the input 1001 is used to decide which model in the archive 1003 needs to be used. The model is selected at 1004 and the k-space data of the input is processed with the selected model. This generated the output MRI image at 1005.

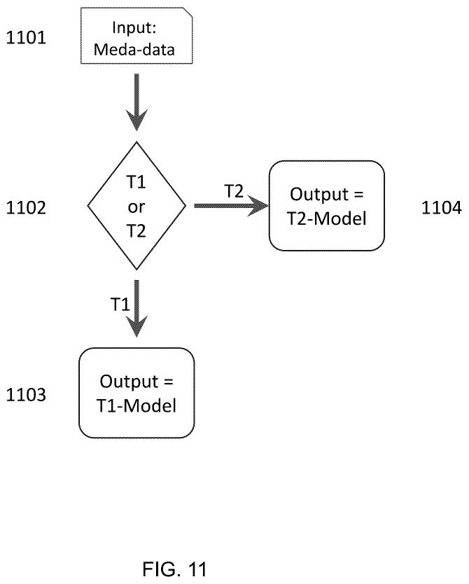

FIG. 11 shows the steps of an example algorithm for inference for T1, T2 and Flair images. This flowchart demonstrates the method for choosing a task-specific model from the model archive based on the meta-data of the input for the specific example of T1 and T2 images. The input meta-data 1101 indicates that the scan is either a T1 or a T2-weighted scan. In 1102, it is checked which one it is. If it is T1, it goes to 1103 and select the model that is tuned for T1-weighted scans. If it is a T2-weighted, it goes to 1104 and select the model that Is tuned for T2-weighted scans. The same algorithm can extend to other applications, types of MRI scans and conditions.

To train the models in the model archive, first, a “master model” model is trained using all the available data. Next, the master model is fine-tuned or further trained for each subcategory of the data (e.g., T1 images) to construct the task-specific models. The master model can be trained on all the data that is available which could include both real data from clinical MRI scans and synthetic or simulated MRI scans. Once the master model is ready, it is further trained on a smaller dataset which only contains samples for a specific application/condition.

For instance, for T1 Axial scans with a 1.5T GE scanner, the master model is fine-tuned on the data samples from 1.5T GE scanners with T1 axial scans. Similarly, this can be done for all the possible applications/conditions to build the full model archive which includes a model for each case.

Table 1 below provides some of the possible different categories of MRI scan tasks or conditions.

TABLE 1
Examples of MRI Scan Categories

| | Field | MRI | Type of | |
|---|---|---|---|---|
| Vendor | Strength | Modality | Scan | Acceleration |
| GE | 1.5T | T1 | Axial | 2-10X |
| Siemens | 3T | T2 | Sagittal | |
| Philips | | Flair | Coronal | |

The model archive involves all the possible combinations in Table 1. In the case of Table 1, this can be 486 models, one for each combination (e.g., Siemens, T2, Sagittal 4×). The model archive may be designed to have a model for each combination/category which can lead to a large archive. Alternatively, the models may be kept only based on some of the attributes (e.g., only MRI modality and scan type which would give 9 models).

Training the models in the model archive can start warm from the master model. This is good particularly if there is not enough data from each sub-category. Alternatively, the training for each model in the archive can start from scratch and can be done independent of the other models in the archive.

Alternatively, this can be done in a cascade. For instance, one can first fine-tune the master model for T1 scans, then break it down to three scan views (i.e., further fine-tune the model trained on T1 scans for Axial, Sagittal and Coronal views), and then fine-tune each one for the field strength and then the vendor. This creates a tree.

The order of the attributes to make the tree/cascade can be any order from the attributes. For instance, it can start with first training for vendors, then the field strength and then the MRI modality, or it can start with the modality then the field strength and then the vendor. The order in this sequence can be treated as a hyperparameter and be optimized for getting the best performance for the models in the archive.

The models can be further fine-tuned for each MRI machine that the models are going to be deployed on. This means that, as part of the deployment process, for each MRI machine that the software is to be deployed on, some sample MRI scans are collected and used to further train and fine-tune the models. This can help further enhance the performance of the models for that specific scanner. Once the fine-tuning is done, a test may also be performed to evaluate the performance of the model, as it may be different from the model that was shipped/deployed.

### Data

Next, a discussion of what kind of data can be used for training the model is provided, along with a high-level description of the ML pipeline.

For a robust training, the system should have as much data as possible, with as diverse of data as possible. The accessible data in the format needed for the models in this disclosure (as described herein) can be very limited as this would require scanning real subjects. Recall that for training the models in this disclosure, we need the raw data from accelerated undersampled scans (MRI-KS-UNDSMPLD) and the ground truth, i.e. the standard MRI images (MRI-IMG-STNRD-FT) and discussed herein, one can get both of these from the raw data of a fully sampled scan (MRI-KS-STNRD). One can supplement the data available to the system with simulated scans and synthetic data.

### Synthetic Data

There are simulators that can simulate the MRI process and provide simulated MRI scans [13]. These simulators simulate the physical processes of the pulses transmitted by the MRI machine and then the response electromagnetic pulses generated by the molecules and tissues in the body and collected by the MRI machine. They can generate realistic MRI scans by mimicking the physical process.

MRI simulators often require some phantoms that the scans are performed on. All the simulations on the same phantom lead to a limited number of samples all of which refer to the same subject. This limits the number of samples generated by the simulators.

For simulation of new samples, the current method can also involve using MRI images (from existing archives) to generate new phantoms (new subjects) and then simulating and creating full scan data (including the raw data) for those phantoms.

Further, for training the models, the method can involve synthesizing data from available MRI images. Note that there are large archives of MRI images available, while access to datasets that include the raw data of the scans are far more limited.

It is possible to synthesize realistic samples (MRI-KS-STNRD) from MRI images. For this, one such approach begins, first, with estimating MRI coil sensitivity maps from the k-space raw data. This can be done for example, for hundred samples of FastMRI training dataset. The central reference region (auto-calibration region) is used to estimate the coil sensitivities with techniques such as ESPRIT. This makes an archive of coil sensitivity maps. Next, magnitude-only MRI images are selected from available MRI image datasets. For instance, we can use multi-vendor, multi-field strength, T1 or T2-weighted brain MRI images. Also, the axial, sagittal, and coronal planes can all be considered. To simulate multi coil acquisition, after multiplication by one a sensitivity map randomly chosen from the sensitivity map archive. Then the images are Fourier transformed to get synthetic k-space. Then, complex Gaussian noise is added according to the noise level of the real data. Variations of the method can be considered for the generation of synthetic data.

A second approach is to use generative ML models to generate samples that are realistic and match the patterns of MRI scans with specific characteristics. For instance, starting with an MRI image, using GAN models, full raw data of the scan (MRI-KS-STNRD) are generated and can be used for training the models.

With the second approach, it is also possible to use generative models such as style-GAN models to add additional information such as pathology. For instance, starting with a healthy MRI image, using a style-GAN, it is also possible to specify a pathology (e.g. cancer) and the model would construct the raw data (MRI-KS-STNRD) samples that match the geometry of the input MRI image but also has the specific input pathology (e.g. cancer). This helps expand the data and cover different conditions that there may not be enough data available for.

One can train the generative models (e.g., GAN) to make samples with specific pathology. For instance, one can make samples with cancerous tumors or dementia. These can be tested using input from radiologists. This can help expand our dataset and cover categories that have fewer samples, which would help with building models that generalize better.

Furthermore, the method can involve augmenting the data. Different augmentations can be included such as motion, rotations, flips, shearing, zoom. Note that the augmentations are not limited to the list here [12]. These can increase the size of the data and help make more robust and generalizable models.

Embodiments of the present disclosure provide a method for training and deployment of machine learning models for reconstruction of MRI images from accelerated MRI scans that can be performed faster than standard (fully-sampled) scans. The method includes: i) new evaluations techniques, metrics and loss functions that are used for training and evaluating the quality of models, ii) new techniques for training the models which combines the advantages of different techniques and also are fine-tuned for specific tasks and as a result perform better, and iii) new techniques for expanding the dataset with synthetic or semi-real samples that can be used to train more robust models.

The methods describe above have been presented in the context of a specific example related to MRI image reconstruction from accelerated MRI scans. However, it can be appreciated that the principles and methods themselves may be used for other medical images (e.g., X-ray, PET or CT scans) and other image processing applications (e.g., motion correction, image restoration or image enhancement). For instance, the benchmarking and evaluation techniques presented herein can be used to assess the quality of images processed for image restoration or motion correction. Or, for example, the method of combining several different techniques using ensemble models can be applied for image restoration or motion correction. Or, for example, the method of training a task/condition specific model that gets better performance can be used for other image processing tasks and other medical imaging modalities.

For simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

Embodiments of the disclosure can be represented as a computer program product stored in a machine-readable medium (also referred to as a computer-readable medium, a processor-readable medium, or a computer usable medium having a computer-readable program code embodied therein). As such, any module or component exemplified herein that executes instructions may include or otherwise have access to computer readable media such as transitory or non-transitory storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory computer readable medium which can be used to store the desired information and which can be accessed by an application, module, or both. Any such computer storage media may be part of the computing environment(s) described herein, any component of or related thereto, etc., or accessible or connectable thereto. Any application or module herein described may be implemented using computer readable/executable instructions that may be stored or otherwise held by such computer readable media.

The steps or operations in the flow charts and diagrams described herein are provided by way of example. There may be many variations to these steps or operations without departing from the principles discussed above. For instance, the steps may be performed in a differing order, or steps may be added, deleted, or modified.

Although the above principles have been described with reference to certain specific examples, various modifications thereof will be apparent to those skilled in the art as having regard to the appended claims in view of the specification as a whole.

### REFERENCES

[1] Sriram, Anuroop, et al. ““End-to-end variational networks for accelerated MRI reconstruction”” Medical Image Computing and Computer Assisted Intervention—MICCAI 2020: 2^{3r}d International Conference, Lima, Peru, Oct. 4-8, 2020, Proceedings, Part II 23. Springer International Publishing, 2020.
[2] Adler, Jonas, and Ozan Öktem. ““Learned primal-dual reconstruction”” IEEE transactions on medical imaging 37.6 (2018): 1322-1332.
[3] Yiasemis, George, et al. ““Recurrent variational network: a deep learning inverse problem Solver applied to the task of accelerated MRI 9 reconstruction”” Proceedings of the IEEE/CVF conference on computer vision and pattern recognition. 2022.
[4] Zbontar, Jure, et al. ““fastMRI: An open dataset and benchmarks for accelerated MRI”” arXiv preprint arXiv: 1811.08839 (2018).
[5] Beauferris, Youssef, et al. ““Multi-coil MRI reconstruction challenge—assessing brain MRI reconstruction models and their generalizability to varying coil configurations”” Frontiers in Neuroscience 16 (2022): 919186.
[6] Wang, Zhou, and Qiang Li. ““Information content weighting for perceptual image quality assessment”” IEEE Transactions on image processing 20.5 (2010): 1185-1198.
[7] Yang, Chun-Ling, Guan-Hao Chen, and Sheng-Li Xie. ““Gradient Information Based Image Quality Assessment”” ACTA ELECTONICA SINICA 35.7 (2007): 1313.
[8] Blaimer, Martin, et al. ““SMASH, SENSE, PILS, GRAPPA: how to choose the optimal method”” Topics in Magnetic Resonance Imaging 15.4 (2004): 223-236.
[9] Lustig, Michael, David Donoho, and John M. Pauly. ““Sparse MRI: The application of compressed sensing for rapid MR imaging”” Magnetic Resonance in Medicine: An Official Journal of the International Society for Magnetic Resonance in Medicine 58.6 (2007): 1182-1195.
[10] Ronneberger, Olaf, Philipp Fischer, and Thomas Brox. ““U-net: Convolutional networks for biomedical image segmentation”” Medical Image Computing and Computer-Assisted Intervention—MICCAI 2015: 18th International Conference, Munich, Germany, Oct. 5-9, 2015, Proceedings, Part III 18. Springer International Publishing, 2015.
[11] Breiman, Leo. “Bagging predictors” Machine learning 24 (1996): 123-140.
[12] Fabian, Zalan, Reinhard Heckel, and Mahdi Soltanolkotabi. ““Data augmentation for deep learning based accelerated MRI reconstruction with limited data”” International Conference on Machine Learning. PMLR, 2021.
[13] Castillo-Passi, Carlos, et al. ““KomaMRI. jl: An open-source framework for general MRI simulations with GPU acceleration”” Magnetic Resonance in Medicine 90.1 (2023): 329-342.
[14] Kyriakides, George, and Konstantinos G. Margaritis. Hands-On Ensemble Learning with Python: Build highly optimized ensemble machine learning models using scikit-learn and Keras. Packt Publishing Ltd, 2019.
[15] Saba, Tanzila. ““Recent advancement in cancer detection using machine learning: Systematic survey of decades, comparisons and challenges”” Journal of Infection and Public Health 13.9 (2020): 1274-1289.
[16] Renieblas, Gabriel Prieto, et al. ““Structural similarity index family for image quality assessment in radiological images”” Journal of medical imaging 4.3 (2017): 035501-035501.
[17] Li, Xiaotong, and Wesley Armour. ““Intensity-Sensitive Similarity Indexes for Image Quality Assessment”” 2022 26th International Conference on Pattern Recognition (ICPR). IEEE, 2022.
[18] D. Temel and G. AlRegib, ““Boosting in image quality assessment”” 2016 IEEE 18th International Workshop on Multimedia Signal Processing (MMSP), Montreal, QC, Canada, 2016, pp. 1-6, doi: 10.1109/MMSP.2016.7813335.
[19] Liu, Anmin, Weisi Lin, and Manish Narwaria. ““Image quality assessment based on gradient similarity”” IEEE Transactions on Image Processing 21.4 (2011): 1500-1512.
[20] Gu, Ke, et al. ““Structural similarity weighting for image quality assessment”” 2013 IEEE International Conference on Multimedia and Expo Workshops (ICMEW). IEEE, 2013.

1. A method of training machine learning models for processing medical images, the method comprising:
obtaining at least one loss function built using at least one evaluation technique to train machine learning models to process medical images;
combining a plurality of image processing techniques using one or more ensemble techniques;
building at least one task-specific model based on the one or more ensemble techniques and using the at least one loss function, to optimally perform on a corresponding specific type of image processing task or for a corresponding specific application or condition; and
providing the at least one task-specific model to a system that performs the specific type of image processing task or the corresponding specific application or condition.

2. The method of claim 1, wherein the at least one evaluation technique comprises one or more of: i) at least one comparison metric; ii) diagnostic performance metrics to measure performance of the machine learning models or image processing task for the corresponding specific diagnostic application; iii) difference metrics to characterize the difference between the ground truth and processed images by building a difference map and measuring the information contained in the difference map; and iv) structural fidelity to measure how well structures are preserved in an image processed by the machine learning models.

3. The method of claim 2, wherein the at least one evaluation technique further comprises at least one quality metric.

4. The method of claim 2, wherein the at least one comparison metric comprises structure Structural Similarity Index Metric (SSIM), informationally weighted SSIM, or Grad-SSIM.

5. The method of claim 2, wherein the at least one evaluation technique is applied to a foreground of images being processed by ignoring a background of the images being processed.

6. The method of claim 1, wherein the one or more ensemble techniques comprises stacking, boosting, or bagging.

7. The method of claim 1, wherein the image processing is optimized over a foreground of images being processed by skipping a background of the images being processed.

8. The method of claim 1 wherein the image processing comprises reconstructing medical images from accelerated image scans.

9. The method of claim 8, wherein the reconstructing utilizes machine learning (ML).

10. The method of claim 8, wherein the image scans correspond to a magnetic resonance imaging (MRI) scan.

11. The method of claim 8, wherein the specific type of image processing task or the specific application or condition comprises task-specific or tailored models for any one or more of: i) an MRI protocol, ii) a specific vendor or MRI machine, iii) an MRI scan view type, iv) a field strength, or v) an identified application or condition category, or vi) different acceleration rate.

12. The method of claim 9, further comprising utilizing synthetic data to augment the training.

13. The method of claim 12, wherein the synthetic data simulates the physical processes of (a) the pulses transmitted by the MRI machine and (b) the response electromagnetic pulses generated by the molecules and tissues in the body and collected by the MRI machine.

14. The method of claim 12, wherein utilizing the synthetic data comprises one or more of the following: i) synthesizing samples using MRI images and coil sensitivity maps from real data with a k-space, ii) using synthetic data that is generated using simulators and phantoms generated from MRI images, iii) using synthetic data that is generated using generative models to generate synthetic samples with raw data from the k-space, iv) using generative models to synthesize samples with a specific pathology or attributes; or v) augmenting existing samples both at a k-space level and an image level.

15. The method of claim 14, wherein the generative models comprise a Generative Adversarial Network (GAN) or style-GAN model.

16. The method of claim 1, wherein the image processing task comprises MRI motion correction or MRI image enhancement.

17. The method of claim 1, further comprising applying the method to a task related to x-ray, CT scan, or PET imaging.

18. The method of claim 1, further comprising applying the method to synthesize new samples for at least one imaging application.

19. A non-transitory computer readable medium comprising computer executable instructions that, when executed, cause a computing device to:
obtain at least one loss function built using at least one evaluation technique to train machine learning models to process medical images;
combine a plurality of image processing techniques using one or more ensemble techniques;
build at least one task-specific model based on the one or more ensemble techniques and using the at least one loss function, to optimally perform on a corresponding specific type of image processing task or for a corresponding specific application or condition; and
provide the at least one task-specific model to a system that performs the specific type of image processing task or the corresponding specific application or condition.

20. A computing device comprising:
a processor; and
memory comprising computer executable instructions that, when executed by the processor, cause the device to:
obtain at least one loss function built using at least one evaluation technique to train machine learning models to process medical images;
combine a plurality of image processing techniques using one or more ensemble techniques;
build at least one task-specific model based on the one or more ensemble techniques and using the at least one loss function, to optimally perform on a corresponding specific type of image processing task or for a corresponding specific application or condition; and
provide the at least one task-specific model to a system that performs the specific type of image processing task or the corresponding specific application or condition.
